(19) United States
(12) Patent Application Publication
(10) Pub. No.: US 2013/0053761 A1
(43) Pub. Date: Feb. 28, 2013

(54) METHOD AND ARRANGEMENT FOR TREATING AN OBJECT WITH A LOW-TEMPERATURE PLASMA

(71) Applicants: Gregor Morfill, Muenchen (DE); Tetsuji Shimizu, Garching (DE); Julia Zimmermann, Muenchen (DE); Yang-Fang Li, Garching (DE)

(72) Inventors: Gregor Morfill, Muenchen (DE); Tetsuji Shimizu, Garching (DE); Julia Zimmermann, Muenchen (DE); Yang-Fang Li, Garching (DE)

(73) Assignee: Max-Planck-Gesellschaft zur Foerderung der Wissenschaften e.V., Muenchen (DE)

(21) Appl. No.: 13/583,862

(22) PCT Filed: Mar. 10, 2011
(86) PCT No.: PCT/EP2011/001176

§ 371 Date: Oct. 9, 2012

(30) Foreign Application Priority Data
Mar. 10, 2010 (EP) PCT/EP2010/001491

(51) Int. Cl. A61L 2/14; B65B 55/02; A61M 37/00; B01J 19/08; A61L 12/02

(52) U.S. Cl. 604/23; 422/23; 422/186.05; 53/425

(57) ABSTRACT

The invention relates to a method and an arrangement for treating an object (2-4, 6) enclosed in an envelope with a low-temperature plasma, particularly for sterilizing and/or disinfecting and/or decontaminating the object, wherein the low-temperature plasma is applied to a surface of the object (2-4, 6), wherein the low-temperature plasma is applied through an envelope (2-4) so that the low-temperature plasma penetrates the envelope (2-4).

### FIELD OF THE INVENTION

The invention relates to a method and an arrangement for treating an object with a low-temperature plasma, particularly for disinfecting and/or sterilizing the object.

### BACKGROUND OF THE INVENTION

The use of a low-temperature plasma or non-thermal plasma for sterilization purposes and for the decontamination of surfaces is disclosed, for example, in WO 2007/031250 A1 and WO 2008/138504 A8. However, these publications provide that the low-temperature plasma is directly applied to the surface to be treated, i.e. without any obstacles for the plasma flow between the plasma source and the surface to be treated. Therefore, the use of the plasma devices disclosed in the afore-mentioned publications is limited to applications in which the surface to be treated is bare so that the low-temperature plasma can be directly applied to the bare surface.

Further, the use of a low-temperature plasma for sterilization purposes is disclosed in JP 2008-183025 A and ETO, Hiroyuki et al.: “Low-temperature sterilization of wrapped materials using flexible sheet-type dielectric barrier discharge”, Applied Physics Letters 93, 221502 (2008). These publications disclose the sterilization of medical instruments inside specific plastic packaging by a low-temperature plasma. However, it should be noted that the plastic packaging disclosed in these publications comprises an integrated electrode for generating the low-temperature plasma inside the packaging. Therefore, this technology requires a specific packaging with an integrated electrode.

### SUMMARY OF THE INVENTION

The inventors have recognized that it is generally possible to apply a low-temperature plasma through an envelope covering the surface to be treated, wherein the disinfection/sterilizing effect of the low-temperature plasma persists even in view of the envelope between the plasma source and the surface to be treated. Therefore, the invention provides that the low-temperature plasma is applied through the envelope: Either the low-temperature plasma is generated within the envelope or it penetrates the envelope.

In contrast to the afore-mentioned publications, the plasma source is separated from the object to be treated, i.e. the plasma source is not integrated into the envelope. Further, there is—with respect to one embodiment—a distance between the plasma source and the object to be treated. Therefore, the invention does not require a specific packaging comprising an integrated electrode for generating the low-temperature plasma.

The envelope can be a fleece as sold by, for example Kimberly-Clark, under the trade names KC100™, KC500™ or Kimguard One-Step™. Alternatively, suitable fleeces are commercially available from the company Johnson & Johnson under the trade names Tyvek Puchor™ or Tyvek Roll™.

In other words, a sustainable disinfection is made possible by generating plasma within a cover or by using a cover which is permeable for the plasma but not for germs, e.g. bacteria, viruses, fungi, spores.

The term low-temperature plasma refers to a non-thermal plasma in which the ions and neutrals are at a low temperature (e.g. near room temperature) while the electrons are much hotter. Further, this term refers to a plasma with a relatively high pressure near atmospheric pressure.

In one embodiment of the invention, the envelope is a wrapping for postal delivery, e.g. a postal envelope or a postal package, while the object to be treated is a pathogen, which is supposedly within the wrapping for postal delivery. The generation of the low-temperature plasma within the wrapping or the application of the low-temperature plasma to the wrapping thus decontaminates any pathogens within the wrapping or both the postal wrapping and any pathogens within the wrapping. For example, the decontamination of postal items by application of a low-temperature plasma would have avoided the fatalities caused by the shipping of anthrax poisoned briefs in the aftermath of the attack on the twin towers in New York City. However, the afore-mentioned decontamination method is not restricted to anthrax spores but it is also suitable for inactivating other pathogens, e.g. a virus, a fungus, a bacterium or any (other) spore.

It should further be noted that the afore-mentioned decontamination method can be routinely accomplished in postal delivery centers, where all postal items are routinely subjected to the plasma treatment. For example, the plasma source can be placed above a conveyor belt on which the postal items are conveyed, wherein the plasma source applies the low-temperature plasma onto the postal items on the conveyor belt thereby decontaminating the content of these postal items.

It should also be noted that it is possible to use a conveyor belt which is at least partially permeable for the low-temperature plasma so that the plasma is generated under the wound dressing or it penetrates both the conveyor belt and the wrapping of the postal item. In such a case, the plasma source can also be positioned below the conveyor belt applying the low-temperature plasma from below.

In another embodiment of the invention, the object to be treated is a wound of a patient, while the envelope is a wound dressing which is placed on the wound. In this embodiment, the low-temperature plasma penetrates the wound dressing and disinfects the wound. Therefore, the invention also encompasses a special and novel wound dressing which is at least partially permeable for the low-temperature plasma so that the low-temperature plasma can penetrate the wound dressing for disinfecting the wound. Further, the wound dressing according to the invention preferably maintains the disinfection of the wound after the application of the low-temperature plasma. This novel type of wound disinfection is advantageous since it is not necessary to remove the wound dressing which is often difficult and painful for the patient.

In yet another embodiment of the invention, the object to be treated is a contact lense while the envelope is a container including the contact lense, wherein the low-temperature plasma is generated within the container or it penetrates the container and sterilizes the contact lense within the container. Therefore, the invention also encompasses a novel container for contact lenses which is characterized in that the container is at least partially permeable for the low-temperature plasma.

It should further be noted that the low-temperature plasma can be used for the elimination or at least deactivation of biofilms and protein accretions.

In yet another embodiment of the invention, the object is a medical instrument or apparatus, particularly a syringe, a surgical instrument or an implant, while the envelope is a disposable packaging containing the object. The low-temperature plasma is generated within the disposable patching or is applied to the disposable packaging and penetrates the disposable packaging thereby disinfecting/sterilizing the medical instrument or apparatus within the disposable packaging. Therefore, the invention also encompasses a novel disposable packaging for a medical instrument or apparatus which is characterized in that it is at least partially permeable for the low-temperature plasma. It should also be noted that the disposable packaging preferably maintains the disinfection/sterilization of the medical instrument or apparatus within the disposable packaging after the application of the low-temperature plasma. In other words, the disposable packaging is preferably permeable for the low-temperature plasma but impermeable for any pathogens, e.g. bacteria, viruses.

In yet another embodiment of the invention, the envelope is a cover which is covering an outlet of the plasma source, wherein the cover protects the plasma source and/or the treated object and/or the operator of the plasma source.

Finally, one embodiment of the invention provides the application of the low-temperature plasma to an object comprising a complex surface contour with shadowed surface areas which are not visible from above the surface. It is generally not possible to disinfect/sterilize or decontaminate such an object with conventional methods, e.g. ultra violet radiation, since the ultra violet radiation used for disinfection, sterilization and/or decontamination does not reach the shadowed surface. However, the low-temperature plasma also reaches the shadowed surface areas of the complex surface contour of the object so that the entire surface of the object is disinfected/sterilized or decontaminated. For example, the low-temperature plasma can be applied to tooth brushes and other body care products and cosmetic items.

The invention and its particular features and advantages will become more apparent from the following detailed description considered with reference to the accompanying drawings.

### BRIEF DESCRIPTION OF THE DRAWINGS

FIG. 1 shows a perspective view of a conveyor belt for postal items which are decontaminated by a plasma source.

FIG. 2 schematically shows the application of a low-temperature plasma to a wound wherein the low-temperature plasma penetrates the wound dressing.

FIG. 3 shows a container for contact lenses, wherein the contact lenses in the container are sterilized by the application of low-temperature plasma.

FIG. 4 shows a perspective view of a disposable packaging including a syringe wherein the syringe is sterilized by a low-temperature plasma.

FIGS. 5A and 5B schematically illustrate a plasma source comprising a cover which is permeable for the plasma.

FIG. 6 illustrates the conventional sterilization of an object with a complex surface contour by application of ultra violet radiation.

FIG. 7 illustrates the sterilization of the object according to FIG. 6 by application of a low-temperature plasma.

FIG. 8 illustrates a modification of FIG. 1 with a tunnel-shaped plasma source.

### DETAILED DESCRIPTION OF THE DRAWINGS

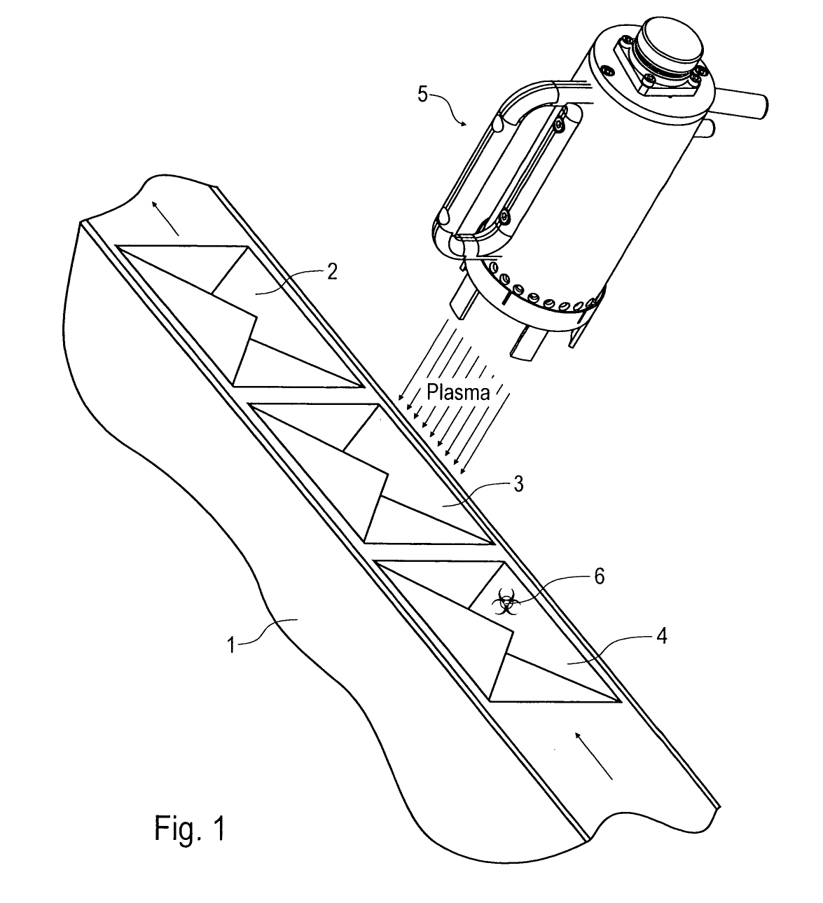

FIG. 1 shows a simplified perspective view of a conveyor belt 1 for conveying postal items 2-4 along a conveyor line. The conveyor belt 1 can be installed, for example, in a postal distribution centre or in a mail room of a company or a government agency, in which the incoming postal items 2-4 are conveyed on the conveyor belt 1 along the conveyor line before further processing of the postal items 2-4.

A plasma source 5 is arranged above the conveyor belt 1 wherein the plasma source 5 discharges a low-temperature plasma onto the postal items 2-4 on the conveyor belt 1.

The plasma source 5 itself is well known in the state of the art and illustrated in WO 2007/031250 A1 so that the content of this publication is incorporated by reference herein. However, it should be noted that other types of plasma sources can be used alternatively, for example the so-called “Venturi-Plaster” as disclosed in WO 2008/138504 A8 or a plasma source comprising a dielectric barrier discharge (DBD) arrangement as disclosed, for example, in PCT/EP2009/007478 so that the content of these publications is also incorporated by reference herein.

The low-temperature plasma discharged by the plasma source 5 penetrates the envelopes of the postal items 2-4 on the conveyor belt 1 and thereby inactivates any pathogens within the envelopes, e.g. an anthrax spore 6 which is schematically shown in the postal item 4.

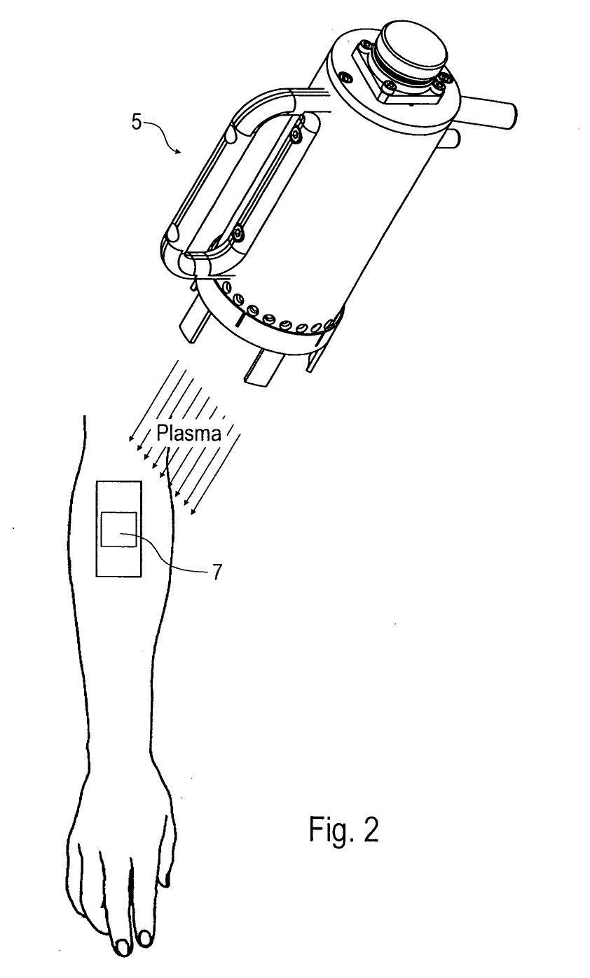

FIG. 2 schematically shows the use of the plasma source 5 for disinfecting a wound which is covered by a wound dressing 7, wherein the low-temperature plasma discharged by the plasma source 5 penetrates the wound dressing 7 and sterilizes the covered wound.

One important feature of the wound dressing 7 is the permeability for the low-temperature plasma generated by the plasma source 5. Another important feature of the novel wound dressing 7 is its ability to maintain the disinfection of the wound after the application of the low-temperature plasma by the plasma source 5. Therefore, it is not necessary to remove and replace the wound dressing 7 for disinfecting the wound.

In all cases, where plasma is used to disinfect, sterilize or decontaminate the content of an envelope, it is possible to use envelopes which block a UV radiation emanating from the plasma source. Especially when disinfecting wounds of a patient the wound dressing 7 preferably will be designed in a way to block UV radiation to protect the patient against said radiation during a disinfection process.

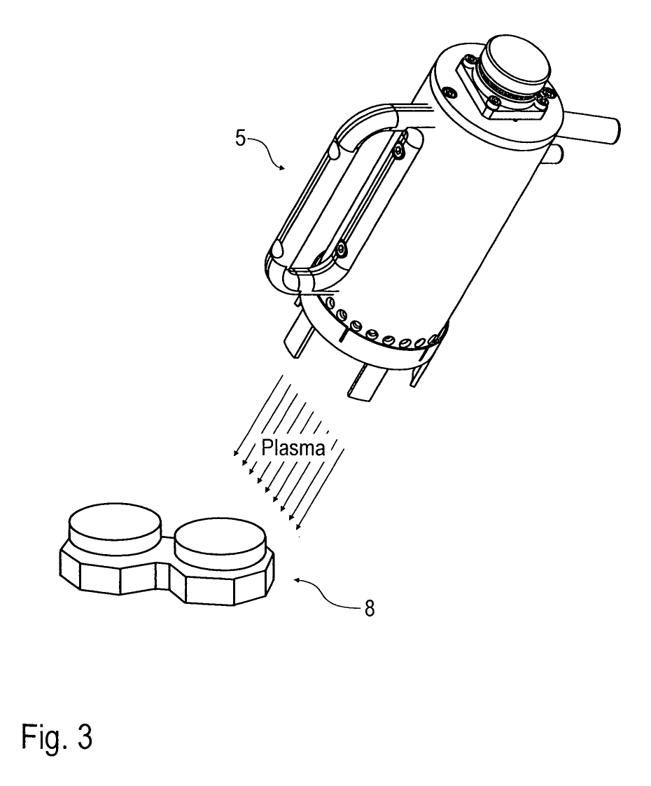

FIG. 3 schematically shows the use of the plasma source 5 for sterilizing contact lenses which are placed in an associated container 8, wherein the container 8 is at least partially permeable for the low-temperature plasma discharged by the plasma source 5 so that the low-temperature plasma penetrates the container 8 and disinfects/sterilizes the contact lenses within the container 8.

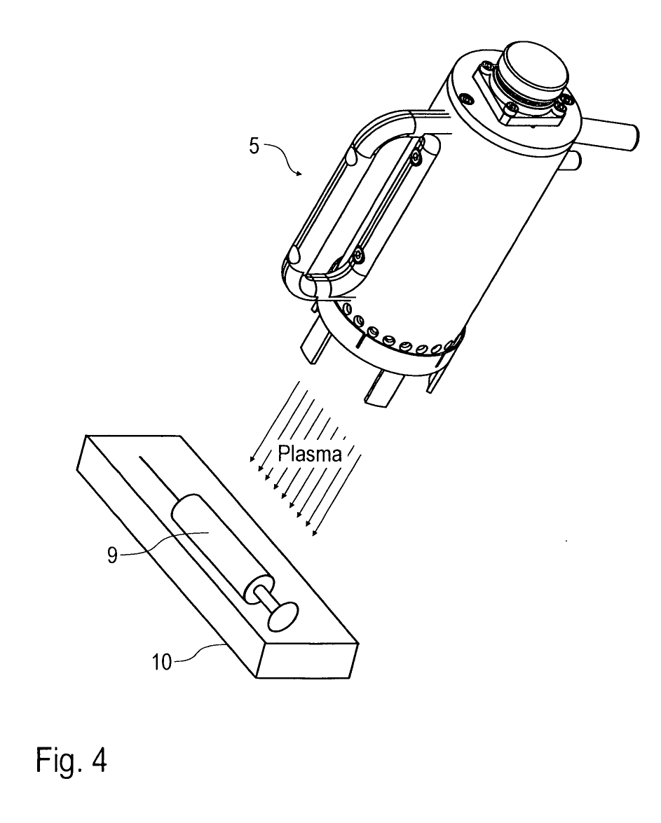

FIG. 4 schematically illustrates the use of the plasma source 5 for disinfecting/sterilizing a syringe 9 in a disposable packaging 10, wherein the disposable packaging 10 is at least partially permeable for the low-temperature plasma discharged by the plasma source. On the other hand, the disposable packaging 10 maintains the sterility of the syringe 9 within the disposable packaging 10 after the application of the low-temperature plasma by the plasma source 5.

Referring to FIG. 4 it is clearly to be seen that not only syringes 9 but also specific articles for babies, i.e. baby bottles, pacifiers or the like may be disinfected, sterilized or decontaminated. As these objects are placed in a disposable packing 10, preferably made of Tyvek, they easily can be transported without being contaminated as the disposable packing 10 maintains the sterility of the objects placed therein.

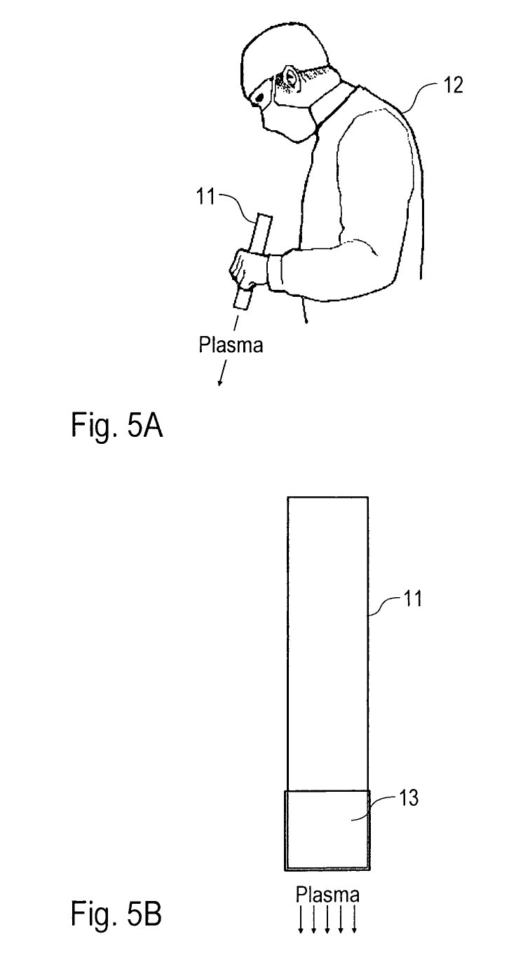

FIGS. 5A and 5B schematically illustrate the use of a similar type of plasma source 11 by a surgeon 12, e.g. for cauterizing tissue.

The plasma source 11 discharges a low-temperature plasma through a cover 13 which protects the surgeon 12 and the patient (not shown) from the low-temperature plasma discharged by the plasma source.

On the one hand, the cover 13 is at least partially permeable for the plasma. On the other hand, the cover 13 is impermeable for any ultra violet radiation generated within the plasma source 11 so that the plasma source 11 substantially emits no ultra violet radiation.

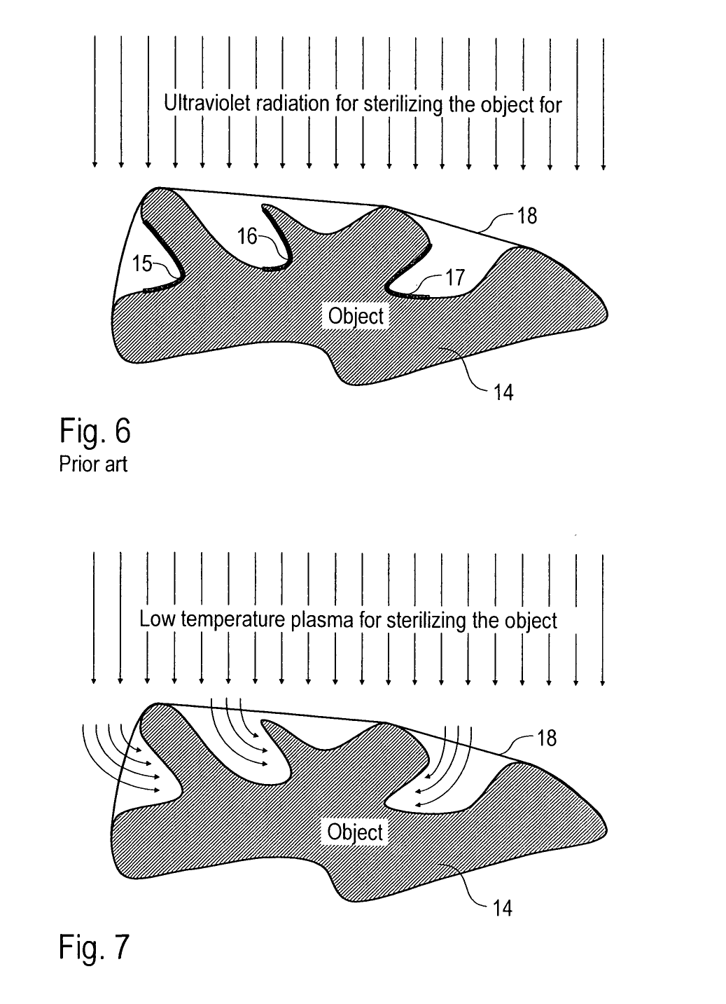

FIG. 6 schematically illustrates the sterilization of an object 14 by application of ultra violet radiation which is well known in the state of the art. In this embodiment, the object 14 comprises a complex surface contour with shadowed surface areas 15-17 which are not visible from above so that the ultra violet radiation does not reach the shadowed surface areas 15-17. Further, the drawing shows that the object 14 is covered by a foil 18 which is well known in the packaging industry.

One major drawback of the afore-mentioned sterilization procedure as shown in FIG. 6 is the fact that the shadowed surface areas 15-17 are substantially not sterilized by the application of the ultra violet radiation.

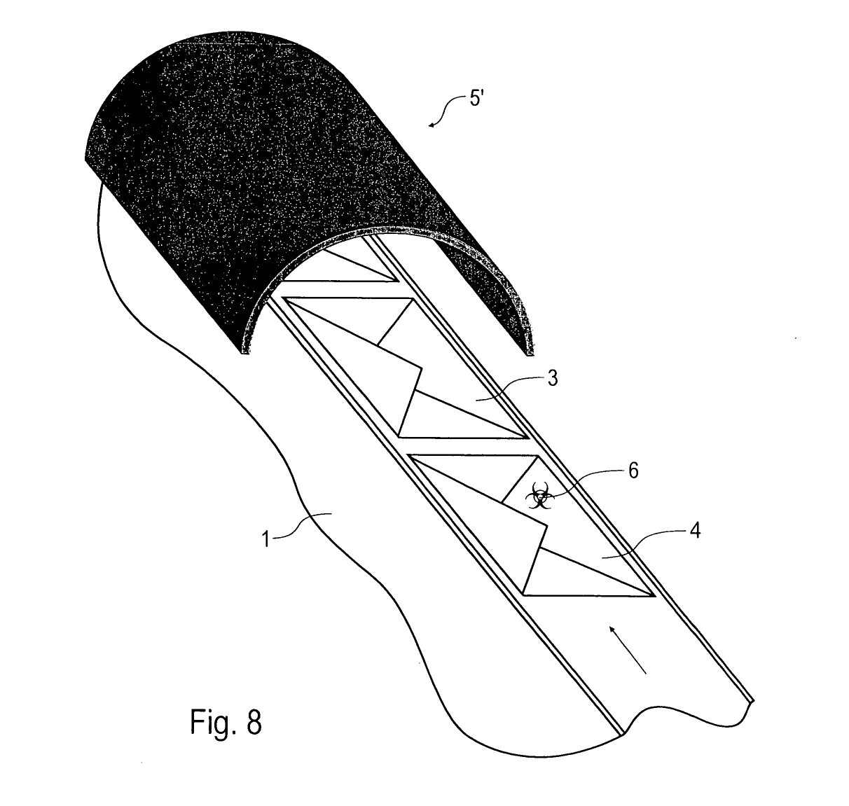

FIG. 7 illustrates the disinfection/sterilization of the object 14 by application of a low-temperature plasma which penetrates the foil 18 and also reaches the shadowed surface areas 15-17 of the object so that the entire surface of the object is sterilized.

FIG. 8 shows a modification of FIG. 1 so that reference is made to the above description.

On characteristic of this embodiment is the design of the plasma source 5′ which forms a tunnel through which the conveyor belt 1 runs. In this embodiment, the plasma source 5′ is a barrier corona discharge (BCD) electrode as disclosed, for example, in PCT/EP2009/001851 which is incorporated by reference herein. It should further be noted that other geometries of the plasma source are possible, as well. For example, the conveyor belt itself can be used as an electrode for generating the plasma.

Referring to the description of the figures it is easily to be seen that an envelope mentioned above may be a bed cloth, a bedding and a mattress. Also clothes, particularly laboratory coats or doctor's overalls are included in the invention: Also clothes are envelopes as mentioned above.

Additionally, the objects to be decontaminated or disinfected/sterilized are not restricted to the ones mentioned above. The method and the arrangement for plasma treating may also be used for decontaminating or disinfecting/sterilizing implantable medical apparatuses, particularly an implantable heart pace maker or an implantable insulin pump. Also body parts of a human being or an animal or food stuffs can be decontaminated or disinfected/sterilized using the method and arrangement mentioned above.

Referring to FIGS. 1 to 8 it is to be seen that the plasma source 5, 5′, 11 is arranged at a distance to the object which is to be decontaminated or disinfected/sterilized by the plasma of the plasma source. In these cases, i.e. arranging the plasma source at a distance to the object, the plasma generated by the plasma source will first reach the outer surface of the envelope, penetrate said envelope to finally reach the object to be disinfected. Arranging the plasma source in a distance to the object, the treatment of said object by plasma will be achieved by the fact that the plasma penetrates the envelope. This envelope may be a wrapping for postal delivery, a wound dressing placed on a wound of a patient, a container including contact lenses, a disposable packing containing a medical instrument or an apparatus or a baby article or a foil including an object which comprises a complex surface contour.

The plasma source providing a low-temperature plasma comprises an electrode. If this electrode is in direct contact with the envelope, the plasma will be produced on the far side of the envelope, i.e. within the envelope. This results in a much faster disinfection/sterilization of object located on the other side of the envelope than in the case of using plasma which has to penetrate the envelope.

Even if the electrode is directly placed on the envelope, it is still possible that plasma will be generated on the surface of the electrode and/or within the envelope itself. This means that in this case the outer surface of the envelope and the envelope itself will be decontaminated or disinfected/sterilized. The plasma being generated on the far side of the envelope will also decontaminate or disinfect/sterilize the inner side of the envelope and the area included within the envelope.

To summarize, if plasma is meant to be generated within an envelope, preferably the electrode of the plasma source will be placed directly on the outer side of the envelope, as this results in the generation of plasma on the far side of the envelope, i.e. within the envelope.

Placing the electrode of a plasma source on the outer side of an envelope to generate plasma within said envelope is advantageous as it is not necessary to use an envelope which allows a diffusive transport of plasma through said envelope. In this case even materials may be used which are plasma tight and prevent any penetration of plasma.

If the conveyor belt depicted in FIG. 8 is used as an electrode for generating the plasma and if an envelope is directly placed on said conveyor belt, it is easily possible to generate plasma within the envelope without the need of a diffusive transport of the plasma through the envelope itself.

In the embodiment depicted in FIGS. 5A and 5B, it is possible to place the cover 13 directly on an electrode of the plasma source 11. In this case the plasma will be generated on the outer surface of the cover 13 and it is possible to use this plasma for directly decontaminating or disinfecting/sterilizing surfaces of objects including wounds of patients. In this case preferably an additional shield is provided to protect the surgeon 12 and/or the patient against excessive plasma and/or ultraviolet radiation generated by the plasma of the plasma source 11.

In the description of the figures and of the embodiments it is said that the plasma is used to eliminate all pathogens. That means that plasma disinfects and/or sterilizes and/or decontaminates all objects and/or envelopes treated with plasma.

In all cases mentioned above, the envelope will be designed in a way, especially when disinfecting wounds of patients, to absorb or preferably block UV radiation to protect the plasma-treated object against this radiation if necessary.

Although the invention has been described with reference to the particular arrangement of parts, features and alike, these are not intended to exhaust all possible arrangements of features, and indeed many other modifications and variations will be ascertainable to those of skill in the art.

### LIST OF REFERENCE NUMERALS

1 Conveyor belt
2-4 Postal item
5 Plasma source
6 Anthrax spore
7 Wound dressing
8 Container
9 Syringe
10 Disposable packaging
11 Plasma source
12 Surgeon
13 Cover
14 Object
15-17 Shadowed surface areas
18 Foil

1. A method for treating an object with a low-temperature plasma, wherein the low-temperature plasma is applied to a surface of the object by a plasma source which is separated from the object, wherein the low-temperature plasma is generated within an envelope or applied through an envelope so that the low-temperature plasma penetrates the envelope.

2. The method according to claim 1, wherein
a) the envelope is a wrapping for postal delivery, and
b) the object is a pathogen, which is supposedly within the wrapping for postal delivery.

3. The method according to claim 1, wherein
a) the envelopes are successively conveyed along a conveyor line, and
b) within the envelopes on the conveyor line successively the low-temperature plasma is generated to inactivate any pathogens within the envelope, or
c) the envelopes on the conveyor line are successively treated with the low-temperature plasma so that the low-temperature plasma penetrates the envelope and inactivates any pathogens within the envelope.

4. The method according to claim 1, wherein
a) the object is a wound of a patient, and
b) the envelope is a wound dressing which is placed on the wound, wherein the low-temperature plasma is generated within the envelope or penetrates the wound dressing and disinfects the wound and sterilizes/disinfects the wound dressing.

5. The method according to claim 4, wherein
a) the wound dressing is at least partially permeable for the low-temperature plasma, and
b) the wound dressing maintains the disinfection of the wound after the application of the low-temperature plasma.

6. The method according to claim 1, wherein the object is a contact lens while the envelope is a container including the contact lens, wherein the low-temperature plasma is generated within the container or penetrates the container and disinfects and/or sterilizes and/or decontaminates the contact lens within the container.

7. The method according to claim 1, wherein
a) the object is a medical instrument, a medical apparatus, or a baby article, and
b) the envelope is a disposable packaging containing the object.

8. The method according to claim 7, wherein
a) the disposable packaging is at least partially permeable for the low-temperature plasma, so that the low-temperature plasma penetrates the disposable packaging and disinfects/sterilizes the object within the disposable packaging, or the plasma is generated within the disposable packaging; and
b) the disposable packaging maintains the disinfection/sterilization of the object within the disposable packaging after the application of the low-temperature plasma.

9. The method according to claim 7, comprising the following steps:
a) Packing the medical apparatus or instrument in the disposable packaging,
b) Sealing the disposable packaging,
c) Applying the low-temperature plasma to the disposable packaging so that the low-temperature plasma is generated within the disposable packaging or penetrates the disposable packaging and sterilizes the medical apparatus or instrument within the disposable packaging.

10. The method according to claim 1, wherein the envelope is a cover which is covering an outlet opening of the plasma source, wherein the cover protects the plasma source nd/or the treated object and/or the operator of the plasma source.

11. The method according to claim 1, wherein the object comprises a complex surface contour with shadowed surface areas which are not visible from above the surface.

12. An arrangement for plasma treating comprising:
a) a plasma source providing a low-temperature plasma,
b) an object which is to be treated with the low-temperature plasma, wherein the plasma source preferably separated from the object,
wherein
c) an envelope at least partially encloses object, wherein the plasma is generated within the envelope and affects the object, or wherein the envelope is permeable for the low-temperature plasma, so that the low-temperature plasma penetrates the envelope and affects the object.

13. The arrangement according to claim 12, wherein the plasma source providing a low-temperature plasma comprises an electrode, designed in a way that it can be placed in direct contact with the envelope.

14. The arrangement according to claim 13, wherein the envelope is selected from a group consisting of:
a) a wrapping for postal delivery,
b) a conveyor belt on which the object is placed,
c) a fleece,
d) a wound dressing covering a wound,
e) a cover which is covering an outlet opening of the plasma source, wherein the cover protects the plasma source and/or the treated object and/or the operator of the plasma source,
f) bedding,
g) a mattress, and
h) clothes.

15. The arrangement according to claim 12, wherein the object is selected from the group consisting of:
a) a medical instrument,
b) a baby article,
c) a medical apparatus,
d) an optical contact lens,
e) foodstuffs,
f) a body part of a human being or an animal,
g) a pathogen; and
h) a wound.

16. The arrangement according to claim 12, wherein
a) the envelope is closer to the plasma source than to the object, or
b) the envelope is closer to the object than to the plasma source.

17. The arrangement according to claim 13, wherein the envelope is a laboratory coat or a doctor's overall.

18. The arrangement according to claim 15, wherein the object is an implantable medical apparatus.

19. The arrangement according to claim 15, wherein the object is a surgical instrument, a syringe, an implantable heart pacemaker, an implantable insulin pump, a virus, a fungus, a spore or a bacterium.

20. The arrangement according to claim 19, wherein the object is an anthrax spore.

21. The method according to claim 2, wherein the envelope is a postal envelope or a postal package.

22. The method according to claim 1, wherein
a) the envelope is closer to the plasma source than to the object, or
b) the envelope is closer to the object than to the plasma source.

23. The method according to claim 1, wherein the object is disinfected, sterilized and/or decontaminated.

24. The method according to claim 2, wherein the pathogen is an anthrax spore.

25. The method according to claim 7, wherein the object is a syringe, a surgical instrument or an implant.
